(19) United States
(12) Patent Application Publication
(10) Pub. No.: US 2006/0128723 A1
(43) Pub. Date: Jun. 15, 2006

(54) Sulptionamide compounds that modulate chemokine receptor activity (CCR4)

(76) Inventors: Antonio Mete, Leicestershire (GB); Barry Teobald, Leicestershire (GB)

Correspondence Address: FISH & RICHARDSON P.C., P.O BOX 1022, MINNEAPOLIS, MN, 55440-1022, US

(21) Appl. No.: 10/559,295

(22) PCT Filed: Jun. 2, 2004
(86) PCT No.: PCT/SE04/00852

§ 371 Date: Dec. 1, 2005

(30) Foreign Application Priority Data
Jun. 4, 2003 (SE) 0301650-8

(51) Int. Cl. A61K 31/497; C07D 409/02

(52) U.S. Cl. 514/255.05; 544/405

(57) ABSTRACT

The invention provides thienylsulphonamides for use in therapy.

The present invention relates to a sulphonamide compound, processes and intermediates used in their preparation, pharmaceutical compositions containing them and their use in therapy.

Chemokines play an important role in immune and inflammatory responses in various diseases and disorders, including asthma and allergic diseases, as well as autoimmune pathologies such as rheumatoid arthritis and atherosclerosis. These small-secreted molecules are a growing superfamily of 8-14 kDa proteins characterised by a conserved four cysteine motif. At the present time, the chemokine superfamily comprises three groups exhibiting characteristic structural motifs, the Cys-X-Cys (C-X-C), Cys-Cys (C-C) and Cys-X₃-Cys (C-X₃-C) families. The C-X-C and C-C families have sequence similarity and are distinguished from one another on the basis of a single amino acid insertion between the NH-proximal pair of cysteine residues. The C-X₃-C family is distinguished from the other two families on the basis of having a triple amino acid insertion between the NH-proximal pair of cysteine residues.

The C-X-C chemokines include several potent chemoattractants and activators of neutrophils such as interleukin-8 (IL-8) and neutrophil-activating peptide 2 (NAP-2).

The C-C chemokines include potent chemoattractants of monocytes and lymphocytes but not neutrophils. Examples include human monocyte chemotactic proteins 1-3 (MCP- 1, MCP-2 and MCP-3), RANTES (Regulated on Activation, Normal T Expressed and Secreted), eotaxin and the macrophage inflammatory proteins 1α and 1β (MIP-1α and MIP-1β), Thymus and Activation Regulated Chemokine (TARC, CCL17) and Macrophage Derived Chemokine (MDC, CCL22). The C-X₃-C chemokine (also known as fractalkine) is a potent chemoattractant and activator of microglia in the central nervous system (CNS) as well as of monocytes, T cells, NK cells and mast cells.

Studies have demonstrated that the actions of chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 for the C-X₃-C family. These receptors represent good targets for drug development since agents which modulate these receptors would be useful in the treatment of disorders and diseases such as those mentioned above.

WO 03/051870 and WO03/059893 discloses a series of sulphonamide compounds said to be useful for treating various diseases. It has now surprisingly been found that a narrow class of compounds generically disclosed in WO 03/051870 exhibit advantageous properties in that they are more potent than derivatives disclosed in WO 03/051870. They also exhibit lower plasma protein binding to human plasma, making these compounds more efficacious for in vivo use.

The present invention therefore provides a compound of formula (I) and pharmaceutically acceptable salts or solvates thereof:

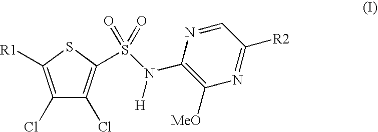

in which:

R¹ is hydrogen or chloro; and

R² is hydrogen, methyl, chloro or bromo.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

Preferably R¹ is hydrogen.

Preferably R² is hydrogen or methyl. Most preferably R² is hydrogen.

Preferred compounds of formula (I) include:

3,4,5-Trichloro-N-(3-methoxypyrazin-2-yl)thiophene-2-sulphonamide

3,4-Dichloro-N-(3-methoxypyrazin-2-yl)thiophene-2-sulphonamide

3,4,5-Trichloro-N-(3-methoxy-5-methylpyrazin-2-yl)thiophene-2-sulphonamide

3,4-Dichloro-N-(3-methoxy-5-methylpyrazin-2-yl)thiophene-2-sulphonamide

N-(5-Bromo-3-methoxypyrazin-2-yl)-3,4-dichlorothiophene-2-sulphonamide

N-(5-Bromo-3-methoxypyrazin-2-yl)-3,4,5-trichlorothiophene-2-sulphonamide

3,4,5-Trichloro-N-(5-chloro-3-methoxypyrazin-2-yl)thiophene-2-sulphonamide.

3,4-Dichloro-N-(5-chloro-3-methoxypyrazin-2-yl)thiophene-2-sulphonamide.

and pharmaceutically acceptable salts and solvates thereof.

Compounds of the invention can be prepared using processes analogous to those of WO 03/051870 or by using the chemistry outlined below:

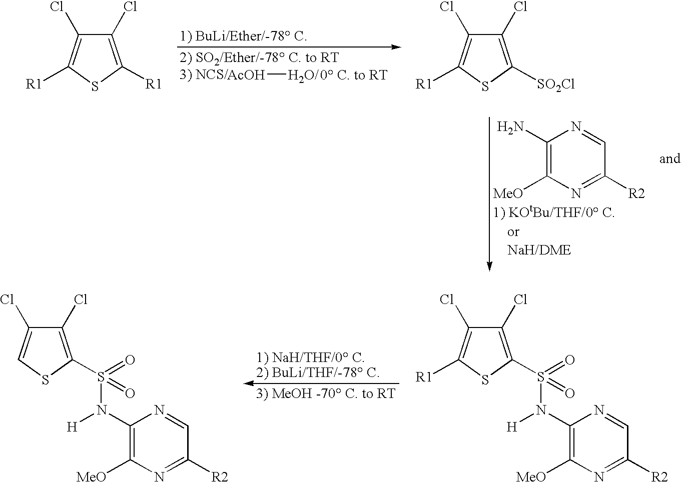

Detailed reaction conditions are given in the examples herein.

It will be appreciated by those skilled in the art that in the processes of the present invention certain functional groups in the starting reagents or intermediate compound may need to be protected by protecting groups. Thus, the preparation of the compound of formula (I) may involve, at an appropriate stage, the removal of one or more protecting groups. The protection and deprotection of functional groups is fully described in ‘Protective Groups in Organic Chemistry’, edited by J. W. F. McOmie, Plenum Press (1973), and ‘Protective Groups in Organic Synthesis’, 3rd edition, T. W. Greene & P. G. M. Wuts, Wiley-Interscience (1999).

The compounds of formula (I) above may be converted to a pharmaceutically acceptable salt or solvate thereof, preferably a basic addition salt such as sodium, potassium, calcium, aluminium, lithium, magnesium, zinc, benzathine, chloroprocaine, choline, diethanolamine, ethanolamine, ethyldiamine, meglumine, tromethamine or procaine, or an acid addition salt such as a hydrochloride, hydrobromide, phosphate, acetate, fumarate, maleate, tartrate, citrate, oxalate, methanesulphonate or p-toluenesulphonate.

The compounds of formula (I) has activity as pharmaceuticals, in particular as modulators of chemokine receptor (especially CCR4) activity, and may be used in the treatment (therapeutic or prophylactic) of conditions/diseases in human and non-human animals which are exacerbated or caused by excessive or unregulated production of chemokines. Examples of such conditions/diseases include:
(1) (the respiratory tract) obstructive airways diseases including chronic obstructive pulmonary disease (COPD); asthma, such as bronchial, allergic, intrinsic, extrinsic and dust asthma, particularly chronic or inveterate asthma (e.g. late asthma and airways hyper-responsiveness); bronchitis; acute, allergic, atrophic rhinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis; seasonal rhinitis including rhinitis nervosa (hay fever) and vasomotor rhinitis; sarcoidosis, farmer's lung and related diseases, fibroid lung and idiopathic interstitial pneumonia;
(2) (bone and joints) gout, rheumatoid arthritis, seronegative spondyloarthropathies (including ankylosing spondylitis, psoriatic arthritis and Reiter's disease), Behcet's disease, Sjogren's syndrome and systemic sclerosis;
(3) (skin) pruritis, scleroderma, otitus, psoriasis, atopical dermatitis, contact dermatitis and other eczmatous dermitides, seborrhoetic dermatitis, Lichen planus, Pemphigus, bullous Pemphigus, Epidermolysis bullosa, urticaria, angiodermas, vasculitides, erythemas, cutaneous eosinophilias, uveitis, Alopecia areata and vernal conjunctivitis, lupus;
(4) (gastrointestinal tract) Coeliac disease, proctitis, eosinopilic gastro-enteritis, mastocytosis, inflammatory bowel diseases such as Crohn's disease, ulcerative colitis, ileitis and enteritis, food-related allergies which have effects remote from the gut, e.g., migraine, rhinitis and eczema;
(5) (central and peripheral nervous system) Neurodegenerative diseases and dementia disorders, e.g. Alzheimer's disease, amyotrophic lateral sclerosis and other motor neuron diseases, Creutzfeldt-Jacob's disease and other prion diseases, HIV encephalopathy (AIDS dementia complex), Huntington's disease, frontotemporal dementia, Lewy body dementia and vascular dementia; polyneuropathies, e.g. Guillain-Barré syndrome, chronic inflammatory demyelinating polyradiculoneuropathy, multifocal motor neuropathy, plexopathies; CNS demyelination, e.g. multiple sclerosis, acute disseminated/haemorrhagic encephalomyelitis, and subacute sclerosing panencephalitis; neuromuscular disorders, e.g. myasthenia gravis and Lambert-Eaton syndrome; spinal diorders, e.g. tropical spastic paraparesis, and stiff-man syndrome: paraneoplastic syndromes, e.g. cerebellar degeneration and encephalomyelitis; CNS trauma; migraine; stroke and correctum diseases such as meningitis
(6) (other tissues and systemic disease) hepatitis, vasculitis, spondyloarthopathies, vaginitis, glomerulonephritis, myositis, atherosclerosis, Acquired Immunodeficiency Syndrome (AIDS), lupus erythematosus, systemic lupus, erythematosus, Hashimoto's thyroiditis, type I diabetes, nephrotic syndrome, eosinophilia fascitis, hyper IgE syndrome, lepromatous leprosy, and idiopathic thrombocytopenia pupura; post-operative adhesions, and sepsis.
(7) (allograft and xenograft rejection) acute and chronic following, for example, transplantation of kidney, heart, liver, lung, bone marrow, skin and cornea; and

chronic graft versus host disease;
(8) Cancer, carcinoma & tumour metastasis, including that of the bladder, breast, colon, kidney, liver, lung, ovary, pancreas, stomach, cervix, thyroid and skin, especially non-small cell lung cancer (NSCLC), malignant melanoma, prostate cancer and squamous sarcoma. Hematopoietic tumors of lymphoid lineage, including acute lymphocytic leukemia, B cell lymphoma and Burketts lymphoma, Hodgkins Lymphoma, Acute Lymphoblastic Leukemia. Hematopoietic tumors of myeloid lineage, including acute and chronic myelogenous leukemias and promyelocytic leukemia. Tumors of mesenchymal origin, including fibrosarcoma and rhabdomyosarcoma, and other tumors, including melanoma, seminoma, tetratocarcinoma, neuroblastoma and glioma.
(9) All diseases that result from a general inbalance of the immune system and resulting in increased atopic inflammatory reactions.
(10) Cystic fibrosis, re-perfusion injury in the heart, brain, peripheral limbs and other organs.
(11) Burn wounds & chronic skin ulcers
(12) Reproductive Diseases (e.g. Disorders of ovulation, menstruation and implantation, Pre-term labour, Endometriosis)
(13) thrombosis
(14) infectious diseases such as HIV infection and other viral infections, bacterial infections.

Thus, the present invention provides a compound of formula (I), or a pharmaceutically-acceptable salt or solvate thereof, as hereinbefore defined for use in therapy.

Preferably the compound of the invention are used to treat diseases in which the chemokine receptor belongs to the CC chemokine receptor subfamily, more preferably the target chemokine receptor is the CCR4 receptor.

Particular conditions which can be treated with the compound of the invention are asthma, rhinitis and inflammatory skin disorders, diseases in which there are raised TARC, MDC or CCR4 levels. It is preferred that the compound of the invention is used to treat asthma and rhinitis, especially asthma.

In a further aspect, the present invention provides the use of a compound of formula (I), or a pharmaceutically acceptable salt or solvate thereof, as hereinbefore defined in the manufacture of a medicament for use in therapy.

In a still further aspect, the present invention provides the use of a compound of formula (I), or a pharmaceutically acceptable salt or solvate thereof, as hereinbefore defined in the manufacture of a medicament for the treatment of human diseases or conditions in which modulation of chemokine receptor activity, particularly CCR4 activity, is beneficial.

In the context of the present specification, the term “therapy” also includes “prophylaxis” unless there are specific indications to the contrary. The terms “therapeutic” and “therapeutically” should be construed accordingly.

The invention still further provides a method of treating a chemokine mediated disease wherein the chemokine binds to a chemokine (especially CCR4) receptor, which comprises administering to a patient a therapeutically effective amount of a compound of formula (I), or a pharmaceutically acceptable salt or solvate thereof, as hereinbefore defined.

The invention also provides a method of treating a respiratory disease, such as asthma and rhinitis, especially asthma, in a patient suffering from, or at risk of, said disease, which comprises administering to the patient a therapeutically effective amount of a compound of formula (I), or a pharmaceutically acceptable salt or solvate thereof, as hereinbefore defined.

For the above-mentioned therapeutic uses the dosage administered will, of course, vary with the compound employed, the mode of administration, the treatment desired and the disorder indicated.

The compound of formula (I) and pharmaceutically acceptable salts and solvates thereof may be used on their own but will generally be administered in the form of a pharmaceutical composition in which the formula (I) compound/salt/solvate (active ingredient) is in association with a pharmaceutically acceptable adjuvant, diluent or carrier. Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I), or a pharmaceutically acceptable salt or solvate thereof, as hereinbefore s defined, in association with a pharmaceutically acceptable adjuvant, diluent or carrier.

The invention further provides a process for the preparation of a pharmaceutical composition of the invention which comprises mixing a compound of formula (I), or a pharmaceutically acceptable salt or solvate thereof, as hereinbefore defined, with a o pharmaceutically acceptable adjuvant, diluent or carrier.

The pharmaceutical compositions may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally. Preferably the compound of the invention is administered orally.

The invention further relates to combination therapies for the treatment of any one of rheumatoid arthritis, osteoarthritis, osteoporosis, psoriasis, inflammatory bowel diseases, COPD, asthma, allergic rhinitis or cancer or the neurodegenerative diseases such as multiple sclerosis, Alzheimer's disease or stroke.

For the treatment of rheumatoid arthritis, the compounds of the invention may be combined with “biological agents” such as TNF-α inhibitors such as anti-TNF monoclonal antibodies (such as Remicade, CDP-870 and Humira) and TNF receptor immunoglobulin molecules (such as Enbrel.reg.). IL-1 receptor antagonist (such as Anakinra) and IL-1 trap, IL- 18 receptor, anti-IL-6 Ab, anti-CD20 Ab, anti-IL-15 Ab and CTLA4Ig.

Suitable agents to be used in combination include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin. The COX-2 inhibitors (such as meloxicam, celecoxib, rofecoxib, valdecoxib and etoricoxib) and the cylco-oxygenase inhibiting nitric oxide donors (CINOD's) and the “disease modifying agents” (DMARDs) such as methotrexate, sulphasalazine, cyclosporine A, lefunomide; ciclesonide; hydroxychloroquine, d-penicillamine, auranofin or parenteral or oral gold.

The present invention still further relates to the combination of a compound of the invention together with a leukotriene biosynthesis inhibitor, 5-lipoxygenase (5-LO) inhibitor or 5-lipoxygenase activating protein (FLAP) antagonist selected from the group consisting of zileuton; ABT-761; fenleuton; tepoxalin; Abbott-79175; Abbott-85761; N-(: substituted)-thiophene-2-alkylsulfonamides; 2,6-di-tert-butylphenol hydrazones; methoxytetrahydropyrans such as Zeneca ZD-2138; the compound SB-210661; pyridinyl substituted 2n cyanonaphthalene compounds such as L-739,010; 2-cyanoquinoline compounds such as L-746,530; indole and quinoline compounds such as MK-591, MK-886, and BAY×1005.

The present invention still further relates to the combination of a compound of the invention together with a receptor antagonists for leukotrienes LTB₄, LTC₄, LTD₄, and LTE₄ selected from the group consisting of the phenothiazin-3-ones such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY×7195.

The present invention still further relates to the combination of a compound of the invention together with a PDE4 inhibitor including inhibitors of the isoform PDE4D.

The present invention still further relates to the combination of a compound of the invention together with a antihistaminic H₁ receptor antagonists including cetirizine, loratadine, desloratadine, fexofenadine, astemizole, azelastine, and chlorpheniramine.

The present invention still further relates to the combination of a compound of the invention together with a gastroprotective H₂ receptor antagonist or the proton pump inhibitors (such as omeprazole)

The present invention still further relates to the combination of a compound of the invention together with an α₁- and α₂-adrenoceptor agonist vasoconstrictor sympathomimetic agent, including propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride.

The present invention still further relates to the combination of a compound of the invention together with anticholinergic agents including ipratropium bromide; tiotropium bromide; oxitropium bromide; pirenzepine; and telenzepine.

The present invention still further relates to the combination of a compound of the invention together with a β₁- to β₄-adrenoceptor agonists including metaproterenol isoproterenol, isoprenaline, albuterol, salbutamol, formoterol, salmeterol, terbutaline, orciprenaline, bitolterol mesylate, and pirbuterol; or methylxanthanines including is theophylline and aminophylline; sodium cromoglycate; or muscarinic receptor (M1, M2, and M3) antagonist.

The present invention still further relates to the combination of a compound of the invention together with other modulators of chemokine receptor function such as CCR1, CCR2, CCR2A, CCR2B, CCR3, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 for the C-X₃-C family.

The present invention still further relates to the combination of a compound of the invention together with an insulin-like growth factor type I (IGF-1) mimetic.

The present invention still further relates to the combination of compound of the invention together with an inhaled glucocorticoid with reduced systemic side effects, including prednisone, prednisolone, flunisolide, triamcinolone acetonide, beclomethasone dipropionate, budesonide, fluticasone propionate, and mometasone furoate.

The present invention still further relates to the combination of a compound of the invention together with (a) tryptase inhibitors; (b) platelet activating factor (PAF) antagonists; (c) interleukin converting enzyme (ICE) inhibitors; (d) IMPDH inhibitors; (e) adhesion molecule inhibitors including VLA-4 antagonists; (f) cathepsins; (g) MAP kinase inhibitors; (h) glucose-6 phosphate dehydrogenase inhibitors; (i) kinin-B₁- and B₂-receptor antagonists; (j) anti-gout agents, e.g., colchicine; (k) xanthine oxidase inhibitors, e.g., allopurinol; (I) uricosuric agents, e.g., probenecid, sulfinpyrazone, and benzbromarone; (m) growth hormone secretagogues; (n) transforming growth factor (TGFβ); (o) platelet-derived growth factor (PDGF); (p) fibroblast growth factor, e.g., basic fibroblast growth factor (bFGF); (q) granulocyte macrophage colony stimulating factor (GM-CSF); (r) capsaicin cream; (s) Tachykinin NK₁ and NK₃ receptor antagonists selected from the group consisting of NKP-608C; SB-233412 (talnetant); and D-4418; and (t) elastase inhibitors selected from the group consisting of UT-77 and ZD-0892 (u) induced nitric oxide synthase inhibitors (iNOS) or (v) chemoattractant receptor-homologous molecule expressed on TH2 cells, (CRTH2 antagonists).

The present invention still further relates to the combination of a compound of the invention together with an inhibitor of matrix metalloproteases (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11).

The compounds of the invention can also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin, induced nitric oxide synthase inhibitors (iNOS inhibitors), COX-2 inhibitors such as celecoxib, valdecoxib, rofecoxib and etoricoxib, and the cylco-oxygenase inhibiting nitric oxide donors (CINOD's) analgesics (such as paracetamol and tramadol), cartilage sparing agents such as diacerein, doxycyline and glucosamine, and intra-articular therapies such as corticosteroids and hyaluronic acids such as hyalgan and synvisc and P2X7 antagonists.

The compounds of the invention can also be used in combination with existing therapeutic agents for the treatment of inflammatory bowel diseases (Ulcerative colitis and Crohn's disease). Suitable agents to be used include sulphasalazine, 5-amino-salicylates, the thiopurines, azathioprine and 6-mecaptorurine and corticosteroids such as budesonide.

The compounds of the present invention may also be used in combination with anticancer agents such as endostatin and angiostatin or cytotoxic drugs such as adriamycin, daunomycin, cis-platinum, etoposide, taxol, taxotere and farnesyl transferase inhibitors, VegF inhibitors, COX-2 inhibitors and antimetabolites such as methotrexate, antineoplastic agents, especially antimitotic drugs including the vinca alkaloids such as vinblastine and vincristine.

The compounds of the invention may also be used in combination with antiviral agents such as Viracept, AZT, aciclovir and famciclovir, and antisepsis compounds such as Valant.

The compounds of the present invention may also be used in combination with cardiovascular agents such as calcium channel blockers, lipid lowering agents such as statins, fibrates, beta-blockers, Ace inhibitors, Angiotensin-2 receptor antagonists and platelet aggregation inhibitors.

The compounds of the present invention may also be used in combination with CNS agents such as antidepressants (such as sertraline), anti-Parkinsonian drugs (such as deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), and anti Alzheimer's drugs such as donepezil, tacrine, COX-2 inhibitors, propentofylline or metryfonate.

The compounds of the present invention may also be used in combination with osteoporosis agents such as roloxifene, droloxifene, lasofoxifene or fosomax and immunosuppressant agents such as FK-506, rapamycin, cyclosporine, azathioprine, and methotrexate.

The compounds of the invention can also be used in combination with existing therapeutic agents for the treatment of cancer. Suitable agents to be used in combination include:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea, gemcitabine and paclitaxel (Taxol®); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);

(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5a-reductase such as finasteride;

(iii) Agents which inhibit cancer cell invasion (for example metalloproteinase inhibitors like marimastat and inhibitors of urokinase plasminogen activator receptor function);

(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;

(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);

(vi) vascular damaging agents such as Combretastatin A4 and compounds disclosed in International Patent Applications WO99/02166, WO00/40529, WO00/41669, WO01/92224, WO02/04434 and WO02/08213;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and

(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The following examples illustrate the invention.

### EXAMPLE 1

### 3,4,5-Trichloro-N-(3-methoxypyrazin-2-yl)thiophene-2-sulphonamide

### a) 3,4,5-Trichlorothiophene-2-sulphonyl chloride

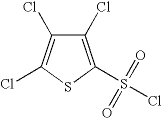

Tetrachlorothiophene (10.0 g) in dry ether (100 ml) was treated with n-butyl lithium (1.3 M solution in hexanes, 35 ml) at −45° C. and stirred for 30 minutes, under an dry nitrogen atmosphere. The solution was cooled to −78° C. and treated with a saturated solution of sulphur dioxide in ether (20 ml). The mixture was stirred at −78° C. for 30 minutes, and then allowed to warm to room temperature over 90 minutes. The resulting suspension was concentrated under reduced pressure and the residue was redissolved in acetic acid (35 ml) and water (35 ml). The solution resulting was treated with N-chlorosuccinimide (6.0 g) portionwise at 0° C., and then stirred at room temperature for 1 hour. The suspended solid was removed by filtration and dried in air to afford the subtitled compound as an orange powder (9.7 g).

m/e 284/286/288 (M⁺), 100%.

MP 56° C.

### b) 3,4,5-Trichloro-N-(3-methoxypyrazin-2-yl)thiophene-2-sulphonamide

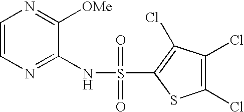

A solution of 3-methoxypyrazin-2-amine (3.5 g) and the product from step (a) (8.0 g) in dry tetrahydrofuran (35 ml) was treated with potassium t-butoxide (1 M solution in tetrahydrofuran, 62 ml), dropwise at 0° C., under a nitrogen atmosphere. The brown suspension was allowed to warm to room temperature over 2 hours and was then poured into 2 M hydrochloric acid and extracted into ethyl acetate. The combined organic extracts were purified on a pad of flash silica which was eluted with ethyl acetate. Concentration of the solvent afforded the titled compound as a brown powder (8.4 g).

m/e 374/376 (M−1)⁻, 100%.

¹H NMR (D6-DMSO) δ 7.79 (1H, br), 7.69 (1H, br), 3.92 (3H, s).

MP 212-214° C.

Elemental Analysis:

Theory: C, 28.85; H, 1.61; N, 11.22%

Found: C, 28.72; H, 1.88; N, 11.23%

### EXAMPLE 2

### 3,4-Dichloro-N-(3-methoxypyrazin-2-yl)thiophene-2-sulphonamide

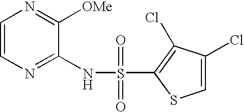

A solution of the product from Example 1, Step (b) (9.6 g) in dry tetrahydrofuran (580 ml) and under a nitrogen atmosphere, was treated with sodium hydride (60% suspension in mineral oil, 1.5 g) at 0° C. and stirred for 10 minutes, then allowed to warm to room temperature for 30 minutes. The solution was cooled to −78° C. and was treated with n-butyl lithium (1.3 M solution in hexanes, 22 ml), dropwise, to give a suspension that was stirred at −78° C. for 60 minutes. Methanol (5 ml) was added, and then the mixture was allowed to warm to room temperature over 90 minutes. The resulting suspension was poured into 2 M hydrochloric acid and was extracted into ethyl acetate. Concentration of the organic phases afforded a brown solid which was purified by flash chromatography on silica using isohexane:ethanol:acetic acid (94:5:1) as solvent. Recrystallisation from isopropanol afforded the titled compound as pale yellow crystals (2.9 g).

m/e 338/340 (M−1)⁻, 100%.

¹H NMR (D6-DMSO+D4−NH₃OH) δ 7.76 (1H, s), 7.33 (1H, d), 7.23 (1H, d), 3.78 (3H, s).

MP 151.5-152.5° C.

Elemental Analysis:

Theory: C, 31.77; H, 2.07; N, 12.35%

Found: C, 31.56; H, 1.99; N, 12.41%

### EXAMPLE 3

### 3,4,5-Trichloro-N-(3-methoxy-5-methylpyrazin-2-yl)thiophene-2-sulphonamide

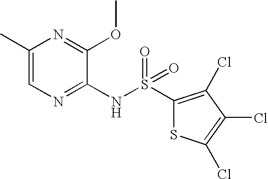

3-methoxy-5-methylpyrazin-2-amine (0.33 g) and the product of Example 1, step (a) were subjected to the procedure described in Example 1, step (b) to afford the product as a pale yellow solid (0.68 g).

m/e 389 (M+1)⁺, 100%.

¹H NMR (CDCl3) δ 7.8 (1H, br), 7.58 (1H, br), 4.01 (3H, s), 2.35 (3H, s).

MP 146-147° C.

Elemental Analysis:

Theory: C, 30.90; H, 2.07; N, 10.81%

Found: C, 31.12; H, 2.00; N, 10.79%

### EXAMPLE 4

### 3,4-Dichloro-N-(3-methoxy-5-methylpyrazin-2-yl)thiophene-2-sulphonamide

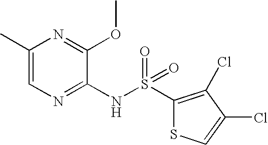

The product from Example 3, (0.54 g) was subjected to the procedure described for Example 2, to give the product as a pale yellow solid, crystallised from ethanol (0.4 g).

m/e 352 (M−1)⁻, 100%.

¹H NMR (D6-DMSO+D4−NH₃OH) ) δ 7.72 (1H, s), 7.18 (1H, s), 3.75 (3H, s), 2.19 (3H, s).

MP 117-118° C.

Elemental Analysis:

Theory: C, 33.91; H, 2.56; N, 11.86%

Found: C, 34.12; H, 2.52; N, 11.79%

### EXAMPLE 5

### N-(5-Bromo-3-methoxypyrazin-2-yl)-3,4-dichlorothiophene-2-sulphonamide, ammonium salt

### a) 3,4-Dichlorothiophene-2-sulphonyl chloride

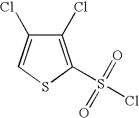

3,4-Dichlorothiophene (1.0 g) in dry tetrahydrofuran (30 ml) was treated with n-butyl lithium (1.3 M solution in hexanes, 5.5 ml) at −78° C. and stirred for 30 minutes, under a dry nitrogen atmosphere. A saturated solution of sulphur dioxide in dry tetrahydrofuran (3 ml) was added, and the mixture was stirred at −78° C. for 75 minutes, and then at room temperature for 2 hours. The resulting suspension was concentrated under reduced pressure and the residue was redissolved in acetic acid (5 ml) and water (5 ml). The solution was treated with N-chlorosuccinimide (0.87 g) portionwise at 0° C., and then stirred at room temperature for 1 hour. The oily mixture was diluted with water and extracted with isohexane. The organic extracts were concentrated under reduced pressure and the residue was purified by flash chromatography on silica using 10% dichloromethane/isohexane as solvent to afford the subtitled compound as a colourless oil (0.55 g).

m/e 250/252/254 (M⁺), 215 (100%).

### b) N-(5-Bromo-3-methoxypyrazin-2-yl)-3,4-dichlorothiophene-2-sulphonamide, ammonium salt

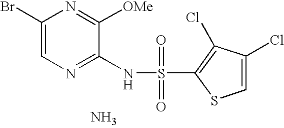

A solution of 3-methoxy-5-bromopyrazin-2-amine (0.20 g) in 1,2-dimethoxyethane (8 ml) was treated with sodium hydride (60% suspension in mineral oil, 0.2 g) at 0° C. and stirred for 15 minutes, under a nitrogen atmosphere. The mixture was warmed to room temperature for 15 minutes and then cooled back to 0° C. and treated with a solution of the product from step (a), (0.3 g) in 1,2-dimethoxyethane (8 ml) over 1 hour. The solution was stirred at room temperature for 1 hour and then poured into dilute aqueous hydrochloric acid and extracted into ethyl acetate. Concentration of the organic extracts afforded a dark gum, which was dissolved in dimethylsulphoxide and purified by reversed phase HPLC on a Waters Symmetry C8 column (19×50 mm) using acetonitrile and 0.1% aqueous ammonium acetate as solvents. The titled compound was obtained as a white powder (0.091 g).

m/e 418/420/422 (M+1)⁺, 100%.

¹H NMR (D6-DMSO) δ 8.00 (1H, s), 7.66 (1H, s), 7.10 (v br m), 3.85 (3H, s).

MP 204° C. decomposes.

### b) N-(5-Bromo-3-methoxypyrazin-2-yl)-3,4-dichlorothiophene-2-sulphonamide, ammonium salt

### EXAMPLE 6

### N-(5-Bromo-3-methoxypyrazin-2-yl)-3,4,5-trichlorothiophene-2-sulphonamide, ammonium salt

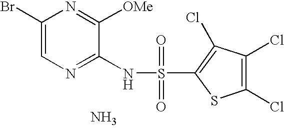

The title compound (0.05 g) was obtained as a second product from the reaction in Example 5.

m/e 452/454/456 (M+1)⁺, 100%.

¹H NMR (D6-DMSO) δ 7.68 (1H, s), 7.09 (v br m), 3.84 (3H, s).

MP 211° C. decomposes.

### EXAMPLE 7

### 3,4,5-Trichloro-N-(5-chloro-3-methoxypyrazin-2-yl)thiophene-2-sulphonamide.

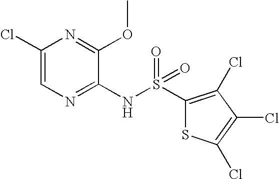

5-Chloro-3-methoxypyrazin-2-amine (0.38 g) and the product of Example 1, step (a) were subjected to the procedure described in Example 1, step (b) to afford the product as a pale yellow solid, crystallised from iso-propyl alcohol (0.47 g).

m/e 408 (M−1)⁻.

¹H NMR (CDCl₃) δ 7.87 (1H, br), 7.77 (1H, s), 4.07 (3H, s).

MP 178-179° C.

Elemental Analysis:

Theory: C, 26.42; H, 1.23; N, 10.27%

Found: C, 26.66; H, 1.21; N, 10.24%

### EXAMPLE 8

### 3,4-Dichloro-N-(5-chloro-3-methoxypyrazin-2-yl)thiophene-2-sulphonamide.

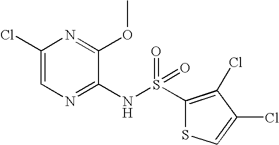

The product from Example 7, (0.4 g) was subjected to the procedure described for Example 2, to give the product as a pale yellow solid, crystallised from ethyl acetate/petrol (0.07 g).

m/e 373 (M−1)⁻.

¹H NMR (CDCl₃) δ 7.89 (1H, br), 7.73 (1H, s), 7.54 (1H, s), 4.07 (3H, s).

MP 116-117 ° C.

Elemental Analysis:

Theory: C, 28.85; H, 1.61; N, 11.22%

Found: C, 29.26; H, 1.58; N, 11.10%

Pharmacological Data

FMAT Whole Cell Binding Assay

Cells

CHO-K1 cells stably expressing the human recombinant CCR4 receptor (Euroscreen; Brussels, Belgium) were cultured in NUT.MIX.F_{—}12(HAM) medium with glutamax-1, containing 10% (v/v) foetal bovine serum and 400 μg ml⁻¹ geneticin.

Cells were harvested at approximately 70% confluence by treatment with a cell dissociation buffer, and seeded at 5×10³ cells/100 μl culture medium into wells of a black Costar clear-bottomed 96-well microtitre plates. Plates were incubated overnight at 37° C. in 5% CO₂ and used the following day.

Assay

Before use, the cell plates were washed twice with 100 μl Hanks balanced salt solution (HBSS). To each well was then added 65 μl of HBSS, 10 μL of 10% DMSO in HBSS±test compound and then 25 μL of 2.8 nM FB-MDC (Applied Biosystems). This fluorescent probe was prepared from a 10 μM stock in 0.08% (v/v) TFA/16% (v/v) acetonitrile, diluted into HBSS.

After two hours incubation in the dark at room temperature, the plates were analysed in an FMAT8100 reader (Applied Biosystems) to measure fluorescence that was associated with binding of FB-MDC to the cells. Compound activity was determined as an pIC₅₀ [log(concentration of compound that results in 50% inhibition)], comparing fluorescence in control and background wells.

Typical Data

All the compounds of the examples have a pIC₅₀ of greater than 5.0.

1. A compound of formula (I) and pharmaceutically acceptable salts or solvates thereof: 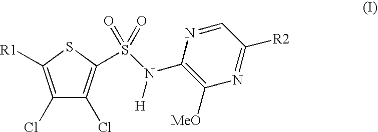
in which:
R¹ is hydrogen or chloro; and
R² is hydrogen, methyl, chloro or bromo.

2. A compound according to claim 1 in which R¹ is hydrogen.

3. A compound according to claim 1 in which R² is hydrogen or methyl.

4. A compound according to claim 1 selected from the group consisting of:
3,4,5-Trichloro-N-(3-methoxypyrazin-2-yl)thiophene-2-sulphonamide
3,4-Dichloro-N-(3-methoxypyrazin-2-yl)thiophene-2-sulphonamide
3,4,5-Trichloro-N-(3-methoxy-5-methylpyrazin-2-yl)thiophene-2-sulphonamide
3,4-Dichloro-N-(3-methoxy-5-methylpyrazin-2-yl)thiophene-2-sulphonamide
N-(5-Bromo-3-methoxypyrazin-2-yl)-3,4-dichlorothiophene-2-sulphonamide
N-(5-Bromo-3-methoxypyrazin-2-yl)-3,4,5-trichlorothiophene-2-sulphonamide
3,4,5-Trichloro-N-(5-chloro-3-methoxypyrazin-2-yl)thiophene-2-sulphonamide and
3,4-Dichloro-N-(5-chloro-3-methoxypyrazin-2-yl)thiophene-2-sulphonamide.
and pharmaceutically acceptable salts and solvates thereof.

5. A pharmaceutical composition comprising a compound of formula (I), or a pharmaceutically acceptable salt or solvate thereof, as claimed in claim 1 in association with a pharmaceutically acceptable adjuvant, diluent or carrier.

6-7. (canceled)

8. A method of treating a chemokine mediated disease wherein the chemokine binds to one or more chemokine receptors, which comprises administering to a patient a therapeutically effective amount of a compound of formula (I), or a pharmaceutically acceptable salt or solvate thereof, as claimed in claim 1.

9. A method according to claim 8 in which the chemokine receptor belongs to the CCR chemokine receptor subfamily.

10. A method according to claim 8 in which the chemokine receptor is the CCR4 receptor.

11. A method of treating an inflammatory disease in a patient suffering from, or at risk of, said disease, which comprises administering to the patient a therapeutically effective amount of a compound of formula (I), or a pharmaceutically acceptable salt or solvate thereof, as claimed in claim 1.

12. A method according to claim 11, wherein the disease is asthma.

13. A process for preparation of a compound of formula (I) 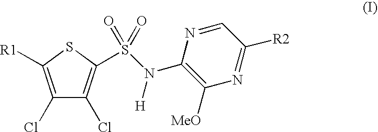
in which:
R¹ is hydrogen and R² is hydrogen, methyl, chloro or bromo, which comprises reacting a compound of formula (I) where R¹ is chloro with a lithiating agent followed by protonation, and optionally thereafter, forming a pharmaceutically acceptable salt or solvate.
